(19) United States
(12) Patent Application Publication
(10) Pub. No.: US 2025/0161406 A1
(43) Pub. Date: May 22, 2025

(54) PREVENTING ALU SINES-MEDIATED PATHOLOGIES

(71) Applicant: UNIVERSITY OF SOUTH FLORIDA, Tampa, FL (US)

(72) Inventor: Hana TOTARY-JAIN, Wesley Chapel, FL (US)

(21) Appl. No.: 18/872,790

(22) PCT Filed: Jun. 6, 2023
(86) PCT No.: PCT/US2023/024552

§ 371 Date: Dec. 6, 2024

Related U.S. Application Data
(60) Provisional application No. 63/349,623, filed on Jun. 7, 2022.

(51) Int. Cl. A61K 38/17; A61P 31/12

(52) U.S. Cl. CPC A61K 38/1709; A61P 31/12

(57) ABSTRACT

A novel method for preventing Alu-mediated interferon related pathologies and excessive Alu-mediated interferon production and inflammatory response is presented. Administration of LIN28B was found to reduce IFN production by binding to Alu RTs and shielding them from binding to dsRNA sensors thus preventing Alu-mediated IFN related pathologies such as viral infections.

### CROSS REFERENCE TO RELATED APPLICATIONS

This application is a nonprovisional of and claims priority to U.S. Provisional Patent Application Ser. No. 63/349,623, entitled “Preventing Alu Sines-Mediated Pathologies”, filed Jun. 7, 2022, the contents of which are hereby incorporated by reference into this disclosure.

### FIELD OF INVENTION

This invention relates to methods of preventing Alu-mediated interferon (IFN) related pathologies. Specifically, the invention provides novel methods of preventing Alu-mediated interferon (IFN) related pathologies through the use of LIN28B, LIN28A, or using their RNA binding domains.

### BACKGROUND OF THE INVENTION

To protect the host against invading viruses, the immune response must be carefully balanced to effectively eliminate pathogens without damaging the host. However, during pregnancy the defense against pathogens may conflict with immunotolerance to the allogeneic fetus and placenta, posing a distinct threat to both the mother and fetus¹. Thus, dynamic alterations in the maternal and fetal immune responses during pregnancy are essential for the success of a species^{16,17}. However, understanding of the mechanisms that regulate the immunological alterations during pregnancy is still lacking.

The placenta provides a powerful physical and immunological barrier that protects the fetus from viral infections⁵. During early placental development, the highly mitotic and self-renewing trophectoderm-derived cytotrophoblasts (CTs) differentiate into either extravillous trophoblasts (EVTs) or fuse through the activity of the endogenous retrovirus fusion protein ERVW-1 (syncytin-1) to form multinucleated syncytiotrophoblasts (STs). The STs form the outer layer of the floating villi and by the end of the first trimester it becomes bathed in maternal blood and regulates the maternal-fetal gas exchange, nutrient uptake, and waste elimination at the interface between the fetal endothelial cells and maternal blood¹⁸. The EVTs at the maternal-fetal interface consist of two major subtypes, the interstitial EVTs that invade the decidua and inner myometrium to anchor the chorionic villi to the uterine wall, and the endovascular EVTs that penetrate and remodel the maternal spiral arteries to facilitate placental perfusion¹⁸.

Viruses can gain access to the decidua and the placenta by either hematogenous transmission or by the lower reproductive tract¹⁷. Invading viruses are recognized by pattern recognition receptors (PRRs) that are found in most cells, including the Toll-like receptors (TLRs) and the retinoic acid-inducible gene I (RIG-I)-like receptors (RLRs)¹⁹. When PRRs recognize pathogen-associated molecular patterns (PAMPs), they initiate intracellular signaling cascades that activate downstream transcription factors such as nuclear factor kB (NFkB) and interferon regulatory factors (IRFs). This results in the production of interferons (IFN), mainly of type I (IFNα and IFNβ) and type III (IFNλ), which are the first line of defense against viral infections. Upon binding to their distinct heterodimeric receptors in an autocrine and/or paracrine manner, type I and type III IFNs trigger the JAK-STAT signaling cascade, which activates hundreds of interferon-stimulated genes (ISGs) that inhibit distinct stages of the viral life cycle eliciting immediate innate and subsequently adaptive immune responses²⁰.

Unlike somatic cells that require PAMPs to mediate IFN induction, human ST cells at the maternal-fetal interface constitutively produce type III IFNs even in the absence of a viral infection³⁻⁶. The release of type III IFNs mediates protection against several RNA and DNA viruses³⁻⁶. However, the molecular mechanisms that constitutively induce type III IFNs in trophoblast cells are not yet defined.

Retrotransposons (RTs) are the most abundant class of the transposable elements that comprise ˜42% of the human genomic sequence²¹. RTs are classified into two groups, those with long terminal repeats (LTRs) such as endogenous retroviruses (ERVs) and those lacking LTRs which include long and short interspersed elements, LINEs, and SINEs, respectively. LTRs and LINEs are autonomous, as they encode their own reverse transcriptase and endonuclease that facilitate their reverse transcription and reintegration into new locations of the host genome. The SINEs on the other hand are non-autonomous and rely on enzymes produced by LINEs for their replication and reintegration. The majority of the human SINEs belong to a single family known as Alu repeats (Alus), named after the internal Alu restriction site found within their ˜300 nucleotide sequence. Alus are primate-specific and comprise ˜11% of the genomic sequence in humans²².

Alu elements are divided into different subfamilies according to key diagnostic nucleotides on them. Therefore, Alu elements sharing the diagnostic nucleotides are grouped into the same subfamily.⁴⁸ Major Alu lineages are AluJ, AluS, and AluY which are distinguished from each other, based on 18 diagnostic nucleotides on their sequences⁴⁷. Among the three major lineages, AluY lineage is the youngest and AluJ lineage is the oldest.⁴⁸

The genomic rearrangements caused by Alu elements could lead to genetic disorders such as hereditary disease, blood disorder, and neurological disorder.

To protect genomic integrity from deleterious insertions of the RTs, differentiated somatic cells under physiological conditions acquired multiple mechanisms to strictly regulate their expression including epigenetic modifications, transcriptional repression, small RNA-silencing, and posttranscriptional processing²¹. Studies have also shown that DICER1 and ADAR play an important role in preventing accumulation of Alus and in producing endogenous siRNA from Alu RTs^{23,24}. In fact, deficiency in DICER1 function has been implicated in age-related macular degeneration, a leading cause of blindness in humans due to Alu toxicity²⁵.

Importantly, during viral infections, RTs regain transcriptional activation²⁶. In particular, the Alu RTs represent the largest class of viral-inducible noncoding RNAs through their internal RNA polymerase III promoter^{27,28}. Viruses that upregulate Alu RNAs include the DNA viruses herpes simplex virus²⁹, adenovirus type 5²⁸ and the positive-sense RNA coronaviruses including SARS-CoV-2³⁰. While much progress has been made in the field of antiviral immunity, the role of Alu RT activation during viral infections is still rudimentary.

In primates, invasive placentation evolved concurrently with the mir-498(96) cistron (also known as MIR-498(46) CISTRON), the largest miRNA cluster in the human genome. This primate-specific cistron spans over 100 kb and contains 46 highly homologous miRNA precursor sequences and 361 RTs, of which 265 are Alu RTs embedded in both the sense (20%) and antisense strands (80%), which have mediated its rapid expansion⁷. The mir-498(46) cistron is preferentially expressed in CTs and STs⁹, where it is epigenetically controlled by imprinting with only the paternal allele transcribed by RNA polymerase II as a single RNA transcript^{31,32}. In the term placenta, mir-498(46) cistron accounts for ˜40% of the total miRNAs⁸, which suggests its pivotal role in regulating trophoblast cell differentiation and function. The inventors as well as others have shown its role in suppressing genes involved in epithelial to mesenchymal transition, which is critical for maintaining the epithelial CTs' stem cell phenotype⁹⁻¹². Moreover, trophoblast-derived exosomes containing specific miRNAs of the mir-498(46) cistron attenuated viral replication by inducing autophagy in recipient cells^{13,14}. Elevated levels of miRNAs of the mir-498(46) cistron in the placenta or in maternal serum correlated with increased risk of development of preeclampsia (PE), a disorder driven by maladaptive immune response³³⁻⁴⁷. While all the research so far has focused on the role of cistron's miRNAs, the role of the Alu RTs that are constitutively transcribed with miRNAs of the mir-498(46) cistron has not been investigated.

Accordingly, what is needed is a method of preventing Alu-mediated IFN related pathologies. The inventors have developed a novel method of preventing Alu-mediated interferon (IFN) related pathologies through the use of proteins LIN28B, LIN28A, fragments thereof, or a combination thereof to reduce IFN production by binding to Alu RTs and shielding them from binding to dsRNA sensors.

### SUMMARY OF INVENTION

To investigate the role of the miRNAs and the Alu RTs of the mir-498(46) cistron in the placenta, the inventors transcriptionally activated the entire cistron using the CRISPR/dCas9 Synergistic Activation Mediator (SAM) system and found an increase in the expression of type III interferon (IFN) and numerous IFN stimulated genes (ISGs), even in the absence of viral infection. To discriminate between the effects of miRNAs and the Alu RTs, the inventors used DICER1 KO 293T cells and found that transcriptional activation of the mir-498(46) cistron increased expression of type III IFNs and ISGs and inhibited viral replication in a miRNA independent manner.

These preliminary results provide the first evidence that the mir-498(46) cistron produces “other” RNA transcripts which are responsible for the induction of these effects. Since ˜50% of the mir-498(46) cistron consists of the highly homologous Alu repeats embedded in both the sense and antisense strands, the inventors believe that the highly expressed mir-498(46) cistron in the syncytiotrophoblasts generates Alu double stranded (ds) RNA, which are responsible for the intrinsic viral resistance of the placenta, and the RNA-binding protein LIN28B acts to counterpoise the Alus dsRNA, and alterations in this balance can lead to either failure to restrict viral spread or to excessive IFN-related pathologies and deleterious impacts on fetal and maternal health.

The inventors have developed a novel method of preventing Alu-mediated interferon (IFN) related pathologies through the use of LIN28B, LIN28A, fragments thereof, or a combination thereof binding to Alus.

In an embodiment, a method of preventing Alu-mediated interferon related pathologies in a subject is presented comprising administering to the subject a therapeutically effective amount of a composition comprising protein LIN28B, LIN28A, fragments thereof, or a combination thereof. The protein LIN28B, LIN28A, fragments thereof, or a combination thereof binds to Alu retrotransposons (Alu RTs) to prevent the ALU RTs from binding to and activating double stranded sensors thus preventing the Alu-mediated interferon related pathologies.

The composition may comprise the protein LIN28B or fragments thereof comprising LIN28B RNA binding sites. In some embodiments, the composition may comprise a plasmid, a synthetic mRNA encoding the protein LIN28B, or LIN28B RNA binding sites.

The composition may be administered parenterally, such as via direct injection intravenously or intramuscularly, or orally to the subject. In some embodiments, the composition is administered site-specifically. In some embodiments, the composition is administered to tissues via nanoparticles.

The Alu-mediated interferon related pathology may be selected from the group consisting of viral infections, bacterial infections, microbial infections, fungal infections, yeast infections, parasitic infections, cancers, autoimmune diseases, inflammatory bowel disease, systemic lupus erythematosus, rheumatoid arthritis, Sjogren's disease, neurodegenerative disorders, neurological disorders, inflammatory disorders, age-related macular degeneration, Geographic atrophy, depression, Parkinson's disease, Alzheimer disease, metabolic disorders, pregnancy complications, and blood disorders. In some embodiments, the Alu-mediated interferon related pathology is a viral infection.

In another embodiment, a method of preventing Alu-mediated interferon (IFN) production and inflammatory response in a subject is presented comprising administering to the subject a therapeutically effective amount of a substance comprising protein LIN28B, LIN28A, fragments thereof, or a combination thereof whereby the protein LIN28B, LIN28A, fragments thereof, or a combination thereof binds to Alu retrotransposons (Alu RTs) to prevent the IFN production.

The composition may comprise the protein LIN28B or fragments thereof comprising LIN28B binding sites. In some embodiments, the composition may comprise a plasmid or synthetic mRNA encoding the protein LIN28B or LIN28B RNA binding sites.

The composition may be administered parenterally, such as via direct injection intravenously or intramuscularly, or orally to the subject. In some embodiments, the composition is administered site-specifically. In some embodiments, the composition is administered to tissues via nanoparticles.

In a further embodiment, a method of preventing viral infection of a cell caused by upregulation of Alu retrotransposons (RTs) in a subject comprising administering to the subject a therapeutically effective amount of a substance comprising protein LIN28B, LIN28A, fragments thereof, or a combination thereof whereby the protein LIN28B, LIN28A, fragments thereof, or a combination thereof binds to Alu retrotransposons (Alu RTs) to prevent the ALU RTs from binding to and activating double stranded sensors thus preventing viral infection.

The composition may comprise the protein LIN28B or fragments thereof comprising LIN28B binding sites. In some embodiments, the composition may comprise a plasmid or synthetic mRNA encoding the protein LIN28B.

The composition may be administered parenterally, such as via direct injection intravenously or intramuscularly, or orally to the subject. In some embodiments, the composition is administered site-specifically. In some embodiments, the composition is administered to tissues via nanoparticles.

The viral infection may be from a virus selected from the group consisting of vesicular stomatitis virus (VSV), Zika virus, respiratory syncytial virus (RSV), severe acute respiratory syndrome CoV-2 (SARS-CoV-2), vaccinia virus, herpes simplex viruses (HSV), Epstein-Barr virus, cytomegalovirus (CMV), and hepatitis B virus. In some embodiments, the virus is SARS-CoV-2.

### BRIEF DESCRIPTION OF THE DRAWINGS

For a fuller understanding of the invention, reference should be made to the following detailed description, taken in connection with the accompanying drawings, in which:

FIG. 1 is a series of images depicting SARS CoV-2 induces Alu RNA in the human lung. Representative images of in situ hybridization for Alu probe (purple) or scramble control of lung biopsy from Covid 19 positive and negative patients. Nuclei were counterstained with nuclear fast red. Original magnification 20×.

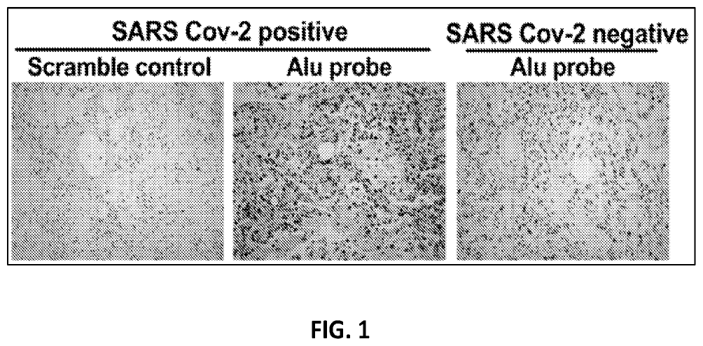

FIG. 2A-F are a series of images depicting the protective role of LIN28B. (A) Representative immunoblot from term placenta lysates from normotensive pregnancies (n=4) and PE pregnancies (n=4) (top) and densitometric analysis for LIN28B normalized to a-tubulin from tissue lysates from normotensive pregnancies (n=15) and PE pregnancies (n=13) (bottom). (B-E) Representative immunoblot, densitometric quantification (B and D), and RT-PCR analysis for LIN28B (B and D) or TNF (C and E) normalized to GAPDH of JEG3 cells transfected with LIN28B shRNA or control shRNA for 72 h (B and C), or of HTR8/SVneo cells transfected with LIN28B encoding plasmids for 72 h (D and E). (F) RT-PCR for IFNL3 normalized to GAPDH in 293T cells transfected with LIN28B shRNA or control shRNA with 759-sgRNA/SAM or BB-sgRNA/SAM control for 72 hr. Results represent means±SEM. *P<0.05 shLIN28B vs. sh-control (B-C) or LIN28B vs. control (D, E).

FIG. 3 is an image depicting LIN28B binds to Alu transcripts. Representative EMSA of AluJb ³²P-labeled probe (lane 1, free probe) and increasing amounts of LIN28B protein (lanes 2 through 6), and in the presence of LIN28B antibody (lane 7), GAPDH antibody (lane 8) or excess of unlabeled AluJb transcripts (lane 9).

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention. The following description is not intended to limit the scope of the present description disclosed herein.

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

Where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed in the invention. The upper and lower limits of these smaller ranges may independently be excluded or included within the range. Each range where either, neither, or both limits are included in the smaller ranges are also encompassed by the invention, subject to any specifically excluded limit in the stated range. Where the stated range includes one or both of the limits, ranges excluding either or both of those excluded limits are also included in the invention.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. As used herein “about” refers to within ±15% of the numerical.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described.

As used in the specification and claims, the singular form “a”, “an” and “the” include plural references unless the context clearly dictates otherwise.

“Patient” is used to describe an animal, preferably a mammal, more preferably a human, to whom treatment is administered, including prophylactic treatment with the compositions of the present invention. The terms “subject” and “patient” are used interchangeably herein.

“Prevention” or “preventing” as used herein refers to any of: halting the effects of an Alu-mediated interferon related pathology, reducing the effects of an Alu-mediated interferon related pathology, reducing the incidence of an Alu-mediated interferon related pathology, reducing the development of an Alu-mediated interferon related pathology, delaying the onset of symptoms of an Alu-mediated interferon related pathology, increasing the time to onset of symptoms of an Alu-mediated interferon related pathology, and reducing the risk of development of an Alu-mediated interferon related pathology.

“Active agent” as used herein is defined as a substance, component or agent that has measurable specified or selective physiological activity when administered to an individual in a therapeutically effective amount. Examples of active agents as used in the present invention include substances which are capable of preventing Alu-mediated interferon related pathologies. At least one active agent is used in the compositions of the present invention. In some embodiments, the active agent is LIN28B protein. In some embodiments, the active agent is LIN28A or a combination of LIN28A and LIN28B. In other embodiments, the active agent is a fragment of LIN28B, LIN28A or a combination of fragments thereof. The fragments may comprise the RNA binding sites thereof.

“Viral vector” as used herein refers to modified viruses used in gene therapy which serve to deliver genetic material into cells. Examples of viral vectors include, but are not limited to, adeno-associated virus, adenovirus, herpes simplex virus, lentivirus, and retrovirus.

“Adeno-associated virus (AAV) vector” as used herein refers to an adeno-associated virus vector that can be engineered for specific functionality in gene therapy. In some instances, the AAV can be a recombinant adeno-associated virus vector, denoted rAAV. Any suitable AAV known in the art can be used, including, but not limited to, AAV2, AAV8, AAV7, AAV6, AAV9, AAV5, AAV1 and AAV4.

“Sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

The term “nucleic acid” as used herein may be double-stranded, single-stranded, or contain portions of both double and single stranded sequence. If the nucleic acid is single-stranded, the sequence of the other strand is also identifiable and thus the definition includes the complement of the sequence disclosed. The nucleic acid may contain modified nucleotides.

“Infection” as used herein refers to the invasion of one or more microorganisms such as bacteria, viruses, fungi, yeast or parasites in the body of a patient in which they are not normally present.

In some embodiments, the infection is a viral infection that is characterized as an RNA virus including, but not limited to, vesicular stomatitis virus (VSV), Zika virus, respiratory syncytial virus (RSV), and coronaviruses such as SARS-CoV2. In some embodiments, the infection is a viral infections that is characterized as an DNA virus including, but not limited to, vaccinia virus, herpes simplex viruses (HSV-1 and -2), Epstein-Barr virus, cytomegalovirus (CMV), and hepatitis B virus.

In some embodiments, the infection may comprise a bacterial infection. The bacterial infection may be caused by any type of infection-inducing bacteria known in the art. For example, in some embodiments, the bacteria includes, but is not limited to, Listeria monocytogenes, Staphylococcus aureus, Streptococcus, Burkholderia pseudomallei, Helicobacter pylori, and Vibrio cholerae.

In some embodiments, the microbial infection is a parasitic, fungal or yeast infection caused by any infection-inducing parasite, fungus, or yeast known in the art including, but not limited to, Toxoplasma gondii, Candida, Cryptococcus, Aspergillus, Histoplasma capsulatum, Coccidioides immitis, C. posadasii, Blastomyces dermatitidis and Pneumocystis jirovecii.

The term “Alu-mediated interferon (IFN) related pathologies” as used herein refers to a disease or disorder in which excessive Alu-mediated IFN production is present leading to an excessive inflammatory response. Examples of Alu-mediated IFN related pathologies includes, but is not limited to, viral infections, bacterial infections, microbial infections, fungal infections, yeast infections, parasitic infections, cancers such as breast and gastric cancers, autoimmune diseases such as multiple sclerosis, inflammatory bowel disease, systemic lupus erythematosus, rheumatoid arthritis, Sjogren's disease, neurodegenerative disorders such as Alzheimer's disease and Parkinson's disease, neurological disorders, Geographic atrophy, depression, Alstrom syndrome, pulmonary arterial hypertension, peeling skin disease, Alport syndrome, Fanconi anemia, Gaucher disease, Pomp disease, Fabry disease, Charcot-Marie Tooth disease, Waardenburg syndrome type 4, hereditary spastic paraplegia, spastic paraplegias, and Peutz-Jeghers syndrome, age-related macular degeneration, metabolic disorders, pregnancy complications, and blood disorders.

The term “pregnancy complication” as used herein refers to any physical and/or mental condition that may affect the health of the pregnant or postpartum subject and/or the baby known in the art. Examples of pregnancy complications include, but are not limited to, preeclampsia, teratogenic effects such as birth defects microcephaly, hearing loss, ocular abnormalities, and/or hepatosplenomegaly, and miscarriage.

In an embodiment, a substance comprising the protein LIN28B, LIN28A, fragments thereof such as RNA binding sites thereof, or a combination thereof may be administered directly to a cell or subject by any means suitable in the art including, but not limited to, injection of the substance into the cell or subject.

In some embodiments, the substance may be a synthetic mRNA encoding the protein LIN28B, LIN28A, fragments thereof such as RNA binding sites thereof, or a combination thereof and may be administered to the subject by any means suitable in the art.

In an embodiment, the substance may be a plasmid, such as a viral vector plasmid, encoding the protein LIN28B, LIN28A, fragments thereof such as RNA binding sites thereof, or a combination thereof, and may be administered to the cell or subject by any means suitable in the art including, but not limited to, electroporation. In some embodiments, the plasmid may be administered to a cell that has been extracted from a subject and the cell containing the plasmid may then be administered to subject.

In an embodiment, the substance may be a viral DNA encoding the protein LIN28B, LIN28A, fragments thereof such as RNA binding sites thereof, or a combination thereof may be administered to the cell or subject by any means suitable in the art including, but not limited to, viral transduction. In some embodiments, the viral DNA encoding the protein LIN28B, LIN28A, fragments thereof such as RNA binding sites thereof, or a combination thereof is administered to a cell, either taken from the subject or grown using cells foreign to the subject, and subsequently the cell is administered to the subject.

In some embodiments, other vector systems including, but not limited to, a lentiviral vector system may be used. The methods described herein for delivery and expression of LIN28B, LIN28A, fragments thereof such as RNA binding sites thereof, or a combination thereof may be applied to alternate viral vector technologies as would be understood by one of ordinary skill in the art.

In some embodiments, the substance comprising the LIN28B protein, LIN28A protein, fragments thereof such as RNA binding sites thereof, or a combination thereof may be formulated into a pharmaceutical composition having a pharmaceutically acceptable carrier. Optionally, the pharmaceutical composition may be comprised of the substance comprising proteins LIN28B, LIN28A, fragments thereof such as RNA binding sites thereof, or a combination thereof in combination with other pharmaceutically active agents or drugs. The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pennsylvania, Mack Publishing Company, 19^{th} ed.) describes formulations which can be used in connection with the subject invention. It is preferred that the pharmaceutically acceptable carrier be one which is chemically inert to the active agent(s) and one which has no detrimental side effects or toxicity under the conditions of use.

The choice of carrier is determined in art by the particular substance comprising the proteins LIN28B, LIN28A, fragments thereof such as RNA binding sites thereof, or a combination thereof as well as the particular method used to administer the substance. For topical formulations, a penetration enhancing agent and/or a suitable wetting agent may be optionally added to the composition. Similarly, suitable additives of any nature in minor proportions may be optionally added such that the additives do not cause any significant deleterious effects.

Oral formulations may comprise a liquid solution such that the therapeutically effective amount of the proteins LIN28B, LIN28A, fragments thereof such as RNA binding sites thereof, or a combination thereof is dissolved in diluents such as water, saline, glycols, oils, alcohols such as benzyl alcohol and polyethylene alcohols (with or without a pharmaceutically acceptable surfactant), and the like in the case of oral liquid preparations such as suspensions, syrups, elixirs and solutions. Oral formulations may also be in the form of capsules, sachets, tablets, lozenges, and troches with each containing a predetermined amount of the active ingredient as a solid or granule. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, macrocrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the substance in a flavor, usually sucrose and acacia or tragacanth, as well as pastilles comprising the substance in an inert base, such as gelatin and glycerin, or sucrose and acacia, emulsions, gels, and the like containing, in addition to, such excipients as are known in the art. Oral formulations may also be in the form of a powder, a suspension in a suitable liquid, or a suitable emulsion.

Formulations for parenteral administration may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In some embodiments, the pharmaceutically acceptable carrier may comprise sterile water, at least in large part, though other ingredients, for example, to aid solubility, may be included. Injectable solutions, for example, may be prepared in which the carrier comprises saline solution, glucose solution or a mixture of saline and glucose solution. In some embodiments, the active agent can be administered in a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-l,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

In some embodiments, the substance comprising the proteins LIN28B, LIN28A, fragments thereof, or a combination thereof may be formulated as an inclusion complex such as cyclodextrin inclusion complexes, liposomes, niosomes, nanoparticles, etc. In some embodiments, the substance is contained within tissue-specific nanoparticles.

“Administration” or “administering” is used to describe the process in which the substances of the present invention, alone or with other substances, are delivered to a patient. The composition may be administered in various ways including parenteral, oral, inhalation, and topical, among others. Each of these conditions may be readily treated using other administration routes of compounds of the present invention to treat a disease or condition. In some embodiments, administration may be site-specific or alternatively, may be made via tissue-specific nanoparticles.

“Parenteral administration” as used herein refers to modes of administration other than enteral and topical administration, usually by injection, and includes, but is not limited to, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

The “therapeutically effective amount” refers to that amount of a therapy (e.g., a therapeutic agent or vector) sufficient to result in the amelioration of an Alu-mediated IFN related pathology or one or more symptoms thereof, prevent advancement of an Alu-mediated IFN related pathology, or cause regression of an Alu-mediated IFN related pathology. In accordance with the present invention, a suitable single dose size is a dose that is capable of preventing or alleviating (reducing or eliminating) a symptom in a patient when administered one or more times over a suitable time period. One of skill in the art can readily determine appropriate single dose sizes for systemic administration based on the size of a mammal and the route of administration.

The amount of the compound in the drug composition will depend on absorption, distribution, metabolism, and excretion rates of the drug as well as other factors known to those of skill in the art. Dosage values may also vary with the severity of the condition to be alleviated. The compounds may be administered once, or may be divided and administered over intervals of time. It is to be understood that administration may be adjusted according to individual need and professional judgment of a person administrating or supervising the administration of the compounds used in the present invention.

The dose of the compounds administered to a subject may vary with the particular composition, the method of administration, and the particular disorder being treated. The dose should be sufficient to affect a desirable response, such as a therapeutic or prophylactic response against a particular disorder or condition. Typically, the attending physician will decide the dosage of the substance with which to treat each individual patient, taking into consideration a variety of factors, such as age, body weight, general health, diet, sex, substance to be administered, route of administration, and the severity of the condition being treated. By way of example and not intending to limit the invention, the dose of the substance can be about 0.001 to about 1000 mg/kg body weight of the subject being treated/day, from about 0.01 to about 10 mg/kg body weight/day, from about 0.01 mg to about 1 mg/kg body weight/day. It is contemplated that one of ordinary skill in the art can determine and administer the appropriate dosage of compounds disclosed in the current invention according to the foregoing considerations.

Dosing frequency for the composition includes, but is not limited to, at least about once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequently. Administration can also be continuous and adjusted to maintaining a level of the compound within any desired and specified range.

The administration of the composition can be extended over an extended period of time, such as from about a month or shorter up to about three years or longer. For example, the dosing regimen can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The compounds used in the present invention may be administered individually, or in combination with or concurrently with one or more other compounds used in other embodiments of the present invention. Additionally, compounds used in the present invention may be administered in combination with or concurrently with preventatives or therapeutics for Alu-mediated interferon related disorders.

The inventors find that LIN28B, LIN28A, fragments thereof such as RNA binding sites thereof, or a combination thereof reduce IFN production by binding to Alu RTs and shielding them from binding to dsRNA sensors thus preventing Alu-mediated IFN related pathologies.

The inventors have recently shown that the RNA binding protein LIN28B, which is the predominant paralog in human placenta, is significantly downregulated in placentas of PE affected pregnancies and plays a putative role in the regulation of trophoblast invasion and inflammation³⁸. LIN28 is known to both regulate and be regulated by the let-7 family of miRNAs. This highly conserved LIN28/let-7 switch governs developmental timing, stem cell self-renewal, cell differentiation, invasion, glucose metabolism, and embryonic growth³⁹. Although LIN28B is known to inhibit miRNA maturation, the inventors previous study showed that LIN28B knockdown reduced the expression of several miRNAs of the mir-498(46) cistron⁴⁰, whereas LIN28B overexpression induces the expression of some miRNAs of the mir-498(46) cistron⁴¹. Moreover, analysis of published PAR-CLIP analysis⁴² and the preliminary results showed that LIN28B can bind to Alu RTs which are dispersed throughout the mir-498(46) cistron. However, the significance of the LIN28B/Alu RNA binding and the regulation of the miRNAs of the mir-498(46) cistron in trophoblast cells have never been investigated.

The results are extended beyond the placenta and the reproductive system, as several cancers highly express this Alu-rich mir-498(46) cistron^{41,43,44}. In fact, amplification of the mir-498(46) cistron and high LIN28 expression has been linked to a distinctly aggressive embryonal brain tumor with multilayered rosettes (EMTR) in children^{45,46}. Moreover, increase in Alu dsRNA due to aberrations in Alu-rich gene expression, methylation, stressful conditions, or viral infections may induce sterile inflammation, such as in autoimmune diseases or exacerbate the inflammatory response to viral infections causing a cytokine storm, respectively. The results lead to a paradigm shift in the understanding of the immunomodulatory role of the Alu RTs thus having tremendous therapeutic implications.

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein. The following examples further illustrate the invention but, of course, should not be construed as in any way limiting its scope.

### EXAMPLE 1

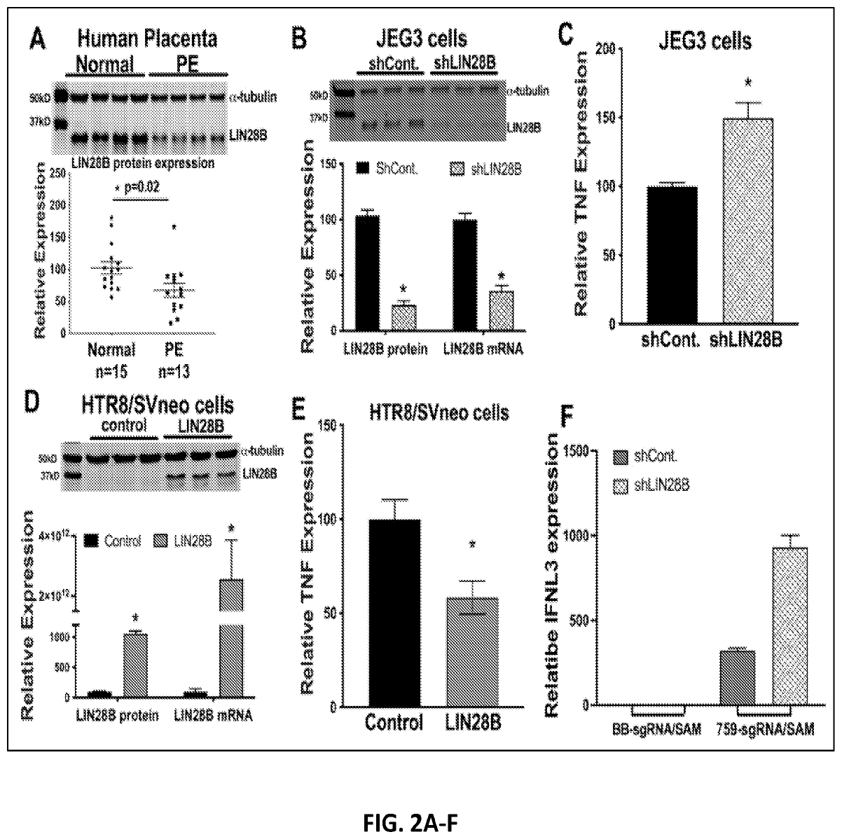

The inventors demonstrate the protective role of LIN28B in preventing excessive Alu-mediated IFN production and inflammatory response. The inventors have recently shown that human placentas express high levels of the RNA binding protein LIN28B and its expression is significantly reduced in placentas from PE-affected pregnancies (FIG. 2A). LIN28B knockdown in JEG3 cells increased TNF expression (FIG. 2B-C), whereas LIN28B overexpression reduced TNF expression (FIG. 2D-E). To investigate the potential role of LIN28B in the mir-498(46)-mediated IFN production, the inventors transfected 293T cells with sh-LIN28B and 759-sgRNA/SAM and after 72 hr, the expression of IFNL3 was determined by RT-PCR. Compared to sh-control, LIN28B silencing increased the IFNL3 expression by more than 2-fold, whereas cells transfected with BB-sgRNA/SAM the expression of IFNL3 was not detected (FIG. 2F). These results provide the first evidence that LIN28B may play a protective role against mir-498(46)-mediated IFN pathologies.

### EXAMPLE 2

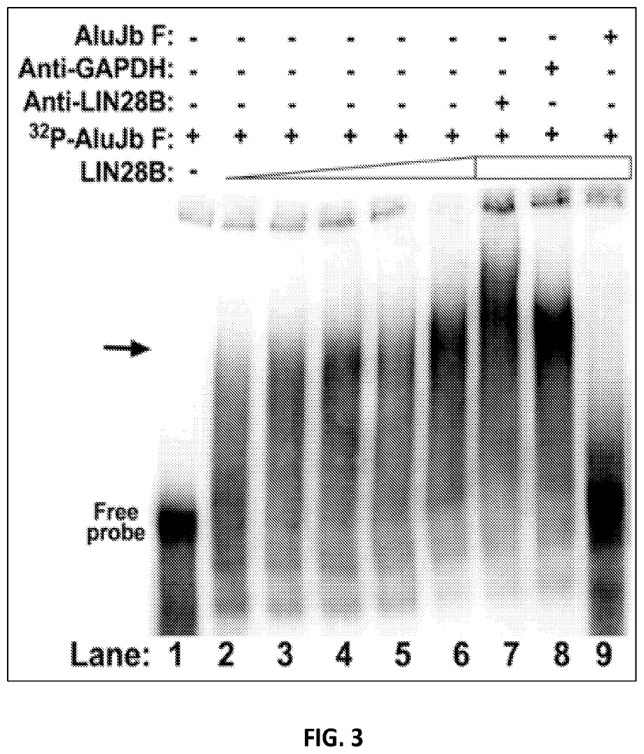

Analyses of previously published photoactivatable ribonucleoside enhanced crosslinking and immunoprecipitation (PAR-CLIP) data show that LIN28B binds numerous Alu repeats located in the mir-498(46) cistron⁴². To directly test whether LIN28B binds to Alu transcripts, the inventors performed electrophoretic mobility shift assay (EMSA) using [³²P]-labeled AluJb probes in vitro transcribed in both the sense and in the antisense directions and found that increasing concentrations of recombinant LIN28B protein increased the binding intensity (arrows). Moreover, a supershift was observed when an antibody against LIN28B was added but not against GAPDH, whereas addition of excess of the unlabeled Alu transcript competed effectively with the labeled probe for LIN28B binding (FIG. 3). Therefore, LIN28B reduces IFN production by binding to Alus and shielding them from binding to the dsRNA sensors, and dysregulation in LIN28B increases the vulnerability to infections and the development of pregnancy complications.

In future experiments, the inventors also establish the LIN28A/B-Alu RNA binding properties; establish the role of LIN28A/B-Alu RNA binding in their recognition by the dsRNA sensor MAD5; establish the role of LIN28A/B-Alu RNA binding in their degradation by DICER1; further investigate the role of LIN28A/B in protecting from the mir-498(46)-mediated IFN pathologies; and investigate the protective potential of LIN28A/B fragments against Alu mediated IFN pathologies.

### EXAMPLE 3 (PROPHETIC)

A 45-year-old female patient at risk for viral infection is parenterally injected with a therapeutically effective amount of a composition comprising LIN28B. The patient does not develop the viral infection.

A 50-year-old male patient at risk for viral infection is orally administered a therapeutically effective amount of a composition comprising LIN28B. The patient does not develop the viral infection.

### CONCLUSION

The inventors find that LIN28B, LIN28A, fragments thereof such as RNA binding sites thereof, or a combination thereof reduce IFN production by binding to Alu RTs and shielding them from binding to dsRNA sensors thus preventing Alu-mediated IFN related pathologies such as viral infections.

### REFERENCES

1. Yockey L J, Lucas C, Iwasaki A. Contributions of maternal and fetal antiviral immunity in congenital disease. Science. 2020; 368(6491):608-12. Epub 2020 May 10.
2. Yockey L J, Iwasaki A. Interferons and Proinflammatory Cytokines in Pregnancy and Fetal Development. Immunity. 2018; 49(3):397-412. Epub 2018 Sep. 21.
3. Bayer A, Lennemann N J, Ouyang Y, Bramley J C, Morosky S, Marques E T, Jr., Cherry S, Sadovsky Y, Coyne C B. Type III Interferons Produced by Human Placental Trophoblasts Confer Protection against Zika Virus Infection. Cell Host Microbe. 2016; 19(5):705-12. Epub 2016 Apr. 14.
4. Corry J, Arora N, Good C A, Sadovsky Y, Coyne C B. Organotypic models of type III interferon-mediated protection from Zika virus infections at the maternal-fetal interface. Proc Natl Acad Sci USA. 2017; 114(35):9433-8. Epub 2017 Aug. 9.
5. Wells A I, Coyne C B. Type III Interferons in Antiviral Defenses at Barrier Surfaces. Trends Immunol. 2018; 39(10):848-58. Epub 2018 Sep. 17.
6. Jagger B W, Miner J J, Cao B, Arora N, Smith A M, Kovacs A, Mysorekar I U, Coyne C B, Diamond M S. Gestational Stage and IFN-lambda Signaling Regulate ZIKV Infection In Utero. Cell Host Microbe. 2017; 22(3):366-76 e3. Epub 2017 Sep. 15.
7. Lehnert S, Van Loo P, Thilakarathne P J, Marynen P, Verbeke G, Schuit F C. Evidence for co-evolution between human microRNAs and Alu-repeats. PLoS One. 2009; 4(2):e4456. Epub 2009 Feb. 12.
8. Williams Z, Ben-Dov I Z, Elias R, Mihailovic A, Brown M, Rosenwaks Z, Tuschl T. Comprehensive profiling of circulating microRNA via small RNA sequencing of cDNA libraries reveals biomarker potential and limitations. Proc Natl Acad Sci USA. 2013; 110(11):4255-60. Epub 2013 Feb. 27.
9. Mong E F, Yang Y, Akat K M, Canfield J, VanWye J, Lockhart J, Tsibris J C M, Schatz F, Lockwood C J, Tuschl T, Kayisli U A, Totary-Jain H. Chromosome 19 microRNA cluster enhances cell reprogramming by inhibiting epithelial-to-mesenchymal transition. Sci Rep. 2020; 10(1):3029. Epub 2020 Feb. 23.
10. Lu Y, Xiao L, Liu Y, Wang H, Li H, Zhou Q, Pan J, Lei B, Huang A, Qi S. MIR517C inhibits autophagy and the epithelial-to-mesenchymal(-like) transition phenotype in human glioblastoma through KPNA2-dependent disruption of TP53 nuclear translocation. Autophagy. 2015; 11(12):2213-32. Epub 2015 Nov. 11.
11. Xie L, Sadovsky Y. The function of miR-519d in cell migration, invasion, and proliferation suggests a role in early placentation. Placenta. 2016; 48:34-7. Epub 2016 Nov. 23.
12. Xie L, Mouillet J F, Chu T, Parks W T, Sadovsky E, Knofler M, Sadovsky Y. C19MC microRNAs regulate the migration of human trophoblasts. Endocrinology. 2014; 155(12):4975-85. Epub 2014 Sep. 12.
13. Delorme-Axford E, Donker R B, Mouillet J F, Chu T, Bayer A, Ouyang Y, Wang T, Stolz D B, Sarkar S N, Morelli A E, Sadovsky Y, Coyne C B. Human placental trophoblasts confer viral resistance to recipient cells. Proc Natl Acad Sci USA. 2013; 110(29):12048-53. Epub 2013 Jul. 3.
14. Krawczynski K, Ouyang Y, Mouillet J F, Chu T, Coyne C B, Sadovsky Y. Unc-13 homolog D mediates an antiviral effect of the chromosome 19 microRNA cluster miR-517a. J Cell Sci. 2020; 134(5). Epub 2020 Oct. 24.
15. Kollmann T R, Kampmann B, Mazmanian S K, Marchant A, Levy O. Protecting the Newborn and Young Infant from Infectious Diseases: Lessons from Immune Ontogeny. Immunity. 2017; 46(3):350-63. Epub 2017 Mar. 23.
16. Silasi M, Cardenas I, Kwon J Y, Racicot K, Aldo P, Mor G. Viral infections during pregnancy. Am J Reprod Immunol. 2015; 73(3):199-213. Epub 2015 Jan. 15.
17. Racicot K, Mor G. Risks associated with viral infections during pregnancy. J Clin Invest. 2017; 127(5):1591-9. Epub 2017 May 2.
18. Knofler M, Haider S, Saleh L, Pollheimer J, Gamage T, James J. Human placenta and trophoblast development: key molecular mechanisms and model systems. Cell Mol Life Sci. 2019; 76(18):3479-96. Epub 2019 May 3.
19. Jensen S, Thomsen A R. Sensing of RNA viruses: a review of innate immune receptors involved in recognizing RNA virus invasion. J Virol. 2012; 86(6):2900-10. Epub 2012 Jan. 20.
20. Schoggins J W. Interferon-Stimulated Genes: What Do They All Do? Annu Rev Virol. 2019; 6(1):567-84. Epub 2019 Jul. 10.
21. Slotkin R K, Martienssen R. Transposable elements and the epigenetic regulation of the genome. Nat Rev Genet. 2007; 8(4):272-85. Epub 2007 Mar. 17.
22. Deininger P. Alu elements: know the SINEs. Genome Biol. 2011; 12(12):236. Epub 2011 Dec. 30.
23. Shiromoto Y, Sakurai M, Qu H, Kossenkov A V, Nishikura K. Processing of Alu small RNAs by DICER/ADAR1 complexes and their RNAi targets. RNA. 2020; 26(12):1801-14. Epub 2020 Aug. 21.
24. Ota H, Sakurai M, Gupta R, Valente L, Wulff B E, Ariyoshi K, Iizasa H, Davuluri R V, Nishikura K. ADAR1 forms a complex with Dicer to promote microRNA processing and RNA-induced gene silencing. Cell. 2013; 153(3):575-89. Epub 2013 Apr. 30.
25. Kaneko H, Dridi S, Tarallo V, Gelfand B D, Fowler B J, Cho W G, Kleinman M E, Ponicsan S L, Hauswirth W W, Chiodo V A, Kariko K, Yoo J W, Lee D K, Hadziahmetovic M, Song Y, Misra S, Chaudhuri G, Buaas F W, Braun R E, Hinton D R, Zhang Q, Grossniklaus H E, Provis J M, Madigan M C, Milam A H, Justice N L, Albuquerque R J, Blandford A D, Bogdanovich S, Hirano Y, Witta J, Fuchs E, Littman D R, Ambati B K, Rudin C M, Chong M M, Provost P, Kugel J F, Goodrich J A, Dunaief J L, Baffi J Z, Ambati J. DICER1 deficit induces Alu RNA toxicity in age-related macular degeneration. Nature. 2011; 471(7338):325-30. Epub 2011 Feb. 8.
26. Capy P, Gasperi G, Biemont C, Bazin C. Stress and transposable elements: co-evolution or useful parasites? Heredity (Edinb). 2000; 85 (Pt 2):101-6. Epub 2000 Sep. 30.
27. Panning B, Smiley J R. Activation of RNA polymerase III transcription of human Alu elements by herpes simplex virus. Virology. 1994; 202(1):408-17. Epub 1994 Jul. 1.
28. Panning B, Smiley J R. Activation of RNA polymerase III transcription of human Alu repetitive elements by adenovirus type 5: requirement for the E1b 58-kilodalton protein and the products of E4 open reading frames 3 and 6. Mol Cell Biol. 1993; 13(6):3231-44. Epub 1993 Jun. 1.
29. Jang K L, Latchman D S. HSV infection induces increased transcription of Alu repeated sequences by RNA polymerase III. FEBS Lett. 1989; 258(2):255-8. Epub 1989 Dec. 4.
30. Yin Y, Liu X Z, He X, Zhou L Q. Exogenous Coronavirus Interacts With Endogenous Retrotransposon in Human Cells. Front Cell Infect Microbiol. 2021; 11:609160. Epub 2021 Mar. 19.
31. Noguer-Dance M, Abu-Amero S, Al-Khtib M, Lefevre A, Coullin P, Moore G E, Cavaille J. The primate-specific microRNA gene cluster (C19MC) is imprinted in the placenta. Hum Mol Genet. 2010; 19(18):3566-82. Epub 2010 Jul. 9.
32. Bortolin-Cavaille M L, Dance M, Weber M, Cavaille J. C19MC microRNAs are processed from introns of large Pol-II, non-protein-coding transcripts. Nucleic Acids Res. 2009; 37(10):3464-73.
33. Hromadnikova I, Kotlabova K, Ivankova K, Krofta L. First trimester screening of circulating C19MC microRNAs and the evaluation of their potential to predict the onset of preeclampsia and IUGR. PLoS One. 2017; 12(2):e0171756. Epub 2017 Feb. 10.
34. Hromadnikova I, Kotlabova K, Ondrackova M, Pirkova P, Kestlerova A, Novotna V, Hympanova L, Krofta L. Expression profile of C19MC microRNAs in placental tissue in pregnancy-related complications. DNA Cell Biol. 2015; 34(6):437-57. Epub 2015 Apr. 1.
35. Hromadnikova I, Dvorakova L, Kotlabova K, Krofta L. The Prediction of Gestational Hypertension, Preeclampsia and Fetal Growth Restriction via the First Trimester Screening of Plasma Exosomal C19MC microRNAs. Int J Mol Sci. 2019; 20(12). Epub 2019 Jun. 21.
36. Miura K, Higashijima A, Murakami Y, Tsukamoto O, Hasegawa Y, Abe S, Fuchi N, Miura S, Kaneuchi M, Masuzaki H. Circulating chromosome 19 miRNA cluster microRNAs in pregnant women with severe pre-eclampsia. J Obstet Gynaecol Res. 2015; 41(10):1526-32. Epub 2015 Aug. 1.
37. Higashijima A, Miura K, Mishima H, Kinoshita A, Jo O, Abe S, Hasegawa Y, Miura S, Yamasaki K, Yoshida A, Yoshiura K, Masuzaki H. Characterization of placenta-specific microRNAs in fetal growth restriction pregnancy. Prenat Diagn. 2013; 33(3):214-22. Epub 2013 Jan. 29.
38. Canfield J, Arlier S, Mong E F, Lockhart J, VanWye J, Guzeloglu-Kayisli O, Schatz F, Magness R R, Lockwood C J, Tsibris J C M, Kayisli U A, Totary-Jain H. Decreased LIN28B in preeclampsia impairs human trophoblast differentiation and migration. FASEB J. 2019; 33(2):2759-69. Epub 2018 Oct. 12.
39. Tsialikas J, Romer-Seibert J. LIN28: roles and regulation in development and beyond. Development. 2015; 142(14):2397-404. Epub 2015 Jul. 23.
40. Canfield J, Arlier S, Mong E F, Lockhart J, VanWye J, Guzeloglu-Kayisli O, Schatz F, Magness R R, Lockwood C J, Tsibris J C M, Kayisli U A, Totary-Jain H. Decreased LIN28B in preeclampsia impairs human trophoblast differentiation and migration. FASEB J. 2018: fj201801163R. Epub 2018 Oct. 12.
41. Mong E F, Akat K M, Canfield J, Lockhart J, VanWye J, Matar A, Tsibris J C M, Wu J K, Tuschl T, Totary-Jain H. Modulation of LIN28B/Let-7 Signaling by Propranolol Contributes to Infantile Hemangioma Involution. Arterioscler Thromb Vasc Biol. 2018; 38(6):1321-32. Epub 2018 May 5.
42. Hafner M, Max K E, Bandaru P, Morozov P, Gerstberger S, Brown M, Molina H, Tuschl T. Identification of mRNAs bound and regulated by human LIN28 proteins and molecular requirements for RNA recognition. RNA. 2013; 19(5):613-26. Epub 2013 Mar. 14.
43. Strub G M, Kirsh A L, Whipple M E, Kuo W P, Keller R B, Kapur R P, Majesky M W, Perkins J A. Endothelial and circulating C19MC microRNAs are biomarkers of infantile hemangioma. JCI Insight. 2016; 1(14):e88856. Epub 2016 Sep. 24.
44. Oberndorfer F, Mullauer L. Genomic alterations in thymoma-molecular pathogenesis? J Thorac Dis. 2020; 12(12):7536-44. Epub 2021 Jan. 16.
45. Hesham D, El-Naggar S. Transcriptomic Analysis Revealed an Emerging Role of Alternative Splicing in Embryonal Tumor with Multilayered Rosettes. Genes (Basel). 2020; 11(9). Epub 2020 Sep. 26.
46. Raghuram N, Khan S, Mumal I, Bouffet E, Huang A. Embryonal tumors with multi-layered rosettes: a disease of dysregulated miRNAs. J Neurooncol. 2020; 150(1):63-73. Epub 2020 Oct. 23.
47. Shen M R, Batzer M A, Deininger P L. Evolution of the master Alu gene(s) J Mol Evol. 1991; 33:311-320.
48. Kim S, Cho C S, Han K, Lee J. Structural Variation of Alu Element and Human Disease. Genomics Inform. 2016 September; 14(3):70-77. doi: 10.5808/GI.2016.14.3.70. Epub 2016 Sep. 30.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall there between. Now that the invention has been described,

What is claimed is:

1. A method of preventing Alu-mediated interferon related pathologies in a subject comprising:
administering to the subject a therapeutically effective amount of a composition comprising protein LIN28B, LIN28A, fragments thereof, or a combination thereof;
wherein the protein LIN28B, LIN28A, fragments thereof, or a combination thereof binds to Alu retrotransposons (Alu RTs) to prevent the ALU RTs from binding to and activating double stranded sensors thus preventing the Alu-mediated interferon related pathologies.

2. The method of claim 1, wherein the fragments thereof are RNA binding sites.

3. The method of claim 2, wherein the composition comprises the protein LIN28B or a fragment thereof comprising the LIN28B RNA binding sites.

4. The method of claim 1, wherein the composition comprises a plasmid or synthetic mRNA encoding the protein LIN28B or LIN28B RNA binding sites.

5. The method of claim 1, wherein the composition is administered parenterally or orally to the subject.

6. The method of claim 1, wherein the composition is administered via direct intravenous or intramuscular injection to the subject.

7. The method of claim 1, wherein the Alu-mediated interferon related pathology is selected from the group consisting of viral infections, bacterial infections, microbial infections, fungal infections, yeast infections, parasitic infections, cancers, autoimmune diseases, inflammatory bowel disease, systemic lupus erythematosus, rheumatoid arthritis, Sjogren's disease, neurodegenerative disorders, neurological disorders, Geographic atrophy, depression, inflammatory disorders, age-related macular degeneration, metabolic disorders, pregnancy complications, and blood disorders.

8. A method of preventing Alu-mediated interferon (IFN) production and inflammatory response in a subject comprising:
administering to the subject a therapeutically effective amount of a substance comprising protein LIN28B, LIN28A, fragments thereof, or a combination thereof;
wherein the protein LIN28B, LIN28A, fragments thereof, or a combination thereof binds to Alu retrotransposons (Alu RTs) to prevent the IFN production.

9. The method of claim 8, wherein the fragments thereof are RNA binding sites.

10. The method of claim 9, wherein the composition comprises the protein LIN28B or a fragment thereof comprising the LIN28B RNA binding sites.

11. The method of claim 8, wherein the composition comprises a plasmid or synthetic mRNA encoding the protein LIN28B.

12. The method of claim 8, wherein the composition is administered parenterally or orally to the subject.

13. The method of claim 8, wherein the composition is administered via direct intravenous or intramuscular injection to the subject.

14. A method of preventing viral infection of a cell caused by upregulation of Alu retrotransposons (RTs) in a subject comprising:
administering to the subject a therapeutically effective amount of a substance comprising protein LIN28B, LIN28A, fragments thereof, or a combination thereof;
wherein the protein LIN28B, LIN28A, fragments thereof, or a combination thereof binds to Alu retrotransposons (Alu RTs) to prevent the ALU RTs from binding to and activating double stranded sensors thus preventing viral infection.

15. The method of claim 14, wherein the fragments thereof are RNA binding sites.

16. The method of claim 15, wherein the composition comprises the protein LIN28B or a fragment thereof comprising the LIN28B RNA binding sites.

17. The method of claim 14, wherein the composition comprises a plasmid or synthetic mRNA encoding the protein LIN28B.

18. The method of claim 14, wherein the composition is administered parenterally or orally to the subject.

19. The method of claim 14, wherein the composition is administered via direct intravenous or intramuscular injection to the subject.

20. The method of claim 14, wherein the viral infection is from a virus selected from the group consisting of vesicular stomatitis virus (VSV), Zika virus, respiratory syncytial virus (RSV), severe acute respiratory syndrome CoV-2 (SARS-CoV-2), vaccinia virus, herpes simplex viruses (HSV), Epstein-Barr virus, cytomegalovirus (CMV), and hepatitis B virus.
